SEQUENCE LISTING (1 sequence)

SEQ ID NO: 1 - protein, 8 aa, Artificial Sequence
  Synthetic peptide
XCYXXTXT

(19) United States
(12) Patent Application Publication
(10) Pub. No.: US 2011/0213193 A1
(43) Pub. Date: Sep. 1, 2011

(54) Magnetic Nanodelivery of Therapeutic Agents Across the Blood Brain Barrier

(75) Inventors: Madhavan P.N. Nair, Coral Gables, FL (US); Zainulabedin M. Saiyed, Miami, FL (US)

(73) Assignee: THE FLORIDA INTERNATIONAL UNIVERSITY BOARD OF TRUSTEES, Miami, FL (US)

(21) Appl. No.: 13/060,713

(22) PCT Filed: Aug. 28, 2009
(86) PCT No.: PCT/US09/55295

§ 371 Date: May 17, 2011

Related U.S. Application Data
(60) Provisional application No. 61/092,491, filed on Aug. 28, 2008.

(51) Int. Cl. A61M 37/00; A61K 9/127; A61K 38/08; A61P 25/00; A61P 25/36; A61P 25/08; A61P 25/16; A61P 35/00; A61P 25/28; A61P 25/18; A61P 25/32; A61P 25/30; B82Y 5/00

(52) U.S. Cl. 600/9; 424/450; 514/21.7; 977/907

(57) ABSTRACT

Liposomes comprising magnetic nanoparticles bound to one or more therapeutic agents are disclosed herein. Also disclosed are methods of delivering a therapeutic agent to a patient across the blood brain barrier using these liposomes, and optionally applying an external magnetic field.

### CROSS-REFERENCE TO RELATED APPLICATIONS

This application claims the benefit of U.S. Provisional Application No. 61/092,491, filed Aug. 28, 2008, the disclosure of which is incorporated by reference in its entirety herein.

### BACKGROUND

The present invention relates to magnetic nanoparticles as a targeting moiety for delivery of therapeutic agents across the blood brain barrier.

It is well established that 100% of large drug molecules and greater than 98% of small molecules do not cross the blood brain barrier (BBB). Despite significant advances in AIDS research and other central nervous system (CNS) disease research (epilepsy, Alzheimer's, Parkinson's, brain tumor, for example), the control or eradication of these diseases remains a challenge due to impenetrability of most drugs across the BBB. Furthermore, abuse of drugs, like opiates, heroin, morphine, methamphetamine, and cocaine, stimulate various CNS cell receptors that promote various neuropathological syndromes in affected individuals. Therefore, development of a delivery system containing multiple drugs and various CNS cell receptor agonists/antagonists that can cross the BBB would provide significant therapeutic benefits.

In the USA, no academic neuroscience program exists that emphasizes BBB transporter biology and brain drug targeting (Pardridge, Pharmaceut. Res., 24(9):1729-1732 (2007)). Nano-sized materials provide a potential carrier system that can cross the BBB and deliver drugs and other agents to the brain, which will have a therapeutic impact on AIDS encephalopathy and CNS diseases, such as epilepsy, Alzheimer's disease, Parkinson's disease, brain tumors, and meningitis, for example.

### SUMMARY

Disclosed herein are magnetic nanoparticles having at least one therapeutic agent bound to the surface of the magnetic nanoparticle. These magnetic nanoparticles are then encapsulated in liposomes. The magnetic liposomes can be used to deliver therapeutic agents to directed areas of a patient's body by external application of a magnetic field, and/or can be used to allow a therapeutic agent to transverse the BBB. The therapeutic agent can be reversibly bound to the surface of the magnetic nanoparticle, by an ionic and/or covalent interaction between the therapeutic agent and the magnetic nanoparticle. In some cases, the covalent interaction is a hydrolysable covalent bond, such as an ester bond, between the therapeutic agent and the magnetic nanoparticle. In various embodiments, the magnetic nanoparticle comprises iron oxide. In some cases, the magnetic nanoparticle has two or more therapeutic agents bound to its surface. In various cases, the liposome can comprise, in addition to the magnetic nanoparticle bound therapeutic agent, another therapeutic agent which is not bound to a magnetic nanoparticle. In some cases, the liposome comprises a first magnetic nanoparticle bound to a first therapeutic agent and a second magnetic nanoparticle bound to a second therapeutic agent. In some specific cases, the therapeutic agent is an antiretroviral or a μ-opioid receptor antagonist. The antiretroviral can be 5-triphosphate-azidothymidine, and the μ-opioid receptor antagonist can be CTOP.

In another aspect, methods of treating a patient by administering a liposome as disclosed herein are described. In some cases, the administration is a parenteral administration, or more specifically, intravenous administration. In various cases, the liposomes provide a sustained release of the therapeutic agent.

In some cases, the patient suffers from Neuro-AIDS, an opiate addiction, or both. In other cases, the patient suffers from a CNS disorder, such as brain carcinoma, epilepsy, Parkinson's disease, Alzheimer's disease, schizophrenia, alcohol addiction, opioid addiction, cocaine addiction, or methamphetamine addiction. In various cases, the patient is further exposed to an external magnetic field to deliver the liposome to a specific part of the body, such as, e.g., exposure about the patient's head, to direct the liposomes to the brain, or to an area of infection or cancer, to direct the liposome to the affected area. In some cases, the liposomes are monitored in the patient's body using magnetic resonance imaging (MRI).

### BRIEF DESCRIPTION OF THE FIGURES

FIG. 1 shows a schematic of the disclosed magnetic liposomes, wherein an antiretroviral agent (azidothymidine 5′-triphosphate AZT-TP; the active form of AZT) and a μ-opioid receptor antagonist (CTOP: D-Phe-Cys-Tyr-D-Trp-Orn-Thr-Pen-Thr; SEQ ID NO: 1) are encapsulated in a liposome along with magnetic nanoparticles. The AZT-TP and CTOP molecules are bound to the surface of the magnetic nanoparticles, and the liposome is modified to include polyethylene glycol (PEG) and RGD-peptide on its outer surface.

FIG. 2 shows a TEM image of magnetic liposomes as disclosed herein.

FIG. 3 shows a scheme for an in vitro model to assess the ability of compositions to cross the BBB by application of an external magnetic field.

FIG. 4 shows a scheme for how a magnetic liposome as disclosed herein transmigrates across the BBB in the presence of an external magnetic field.

FIG. 5 shows a binding isotherm for AZT-TP-bound magnetic nanoparticles.

FIG. 6 shows a binding isotherm for CTOP-bound magnetic nanoparticles.

FIG. 7 shows inhibition of p24 antigen by AZT-TP-bound magnetic nanoparticles.

FIG. 8 shows inhibition of HIV-LTR expression by AZT-TP-bound magnetic nanoparticles.

FIG. 9 shows transmigration of magnetic nanoparticle-loaded monocytes across an in vitro BBB model.

FIG. 10 shows a microscopic image of monocytes loaded with fluorescent magnetic liposomes.

### DETAILED DESCRIPTION

Disclosed herein are magnetic nanoparticles having therapeutic agents bound thereto which can be targeted for delivery across the blood brain barrier (BBB) by incorporation in liposomes. Further disclosed are methods of using these magnetic liposomes for targeting of therapeutic agents to disease sites in the body. The magnetic nanoparticles disclosed herein optionally can be bound to two or more different therapeutic agents belonging to the same or different classes of drugs, e.g., antiretroviral agents, such as nucleoside reverse transcriptase inhibitor (NRTI), non-nucleoside reverse transcriptase inhibitor (NNRTI), and protease inhibitor (PI); other BBB non-penetrating drugs, such as neuropeptides like vasoactive intestinal peptide (VIP), brain derived neurotropic factor (BDNF), fibroblast growth factor-2 (FGF-2), alvimopan, and doxorubicin; CNS cell based receptor agonists, antagonists, and drug abuse receptor agonists and antagonists against μ/κ/δ/ε receptors, such as DPDPE, biphalins, CTOP, and DIPP[Ψ]-amide.

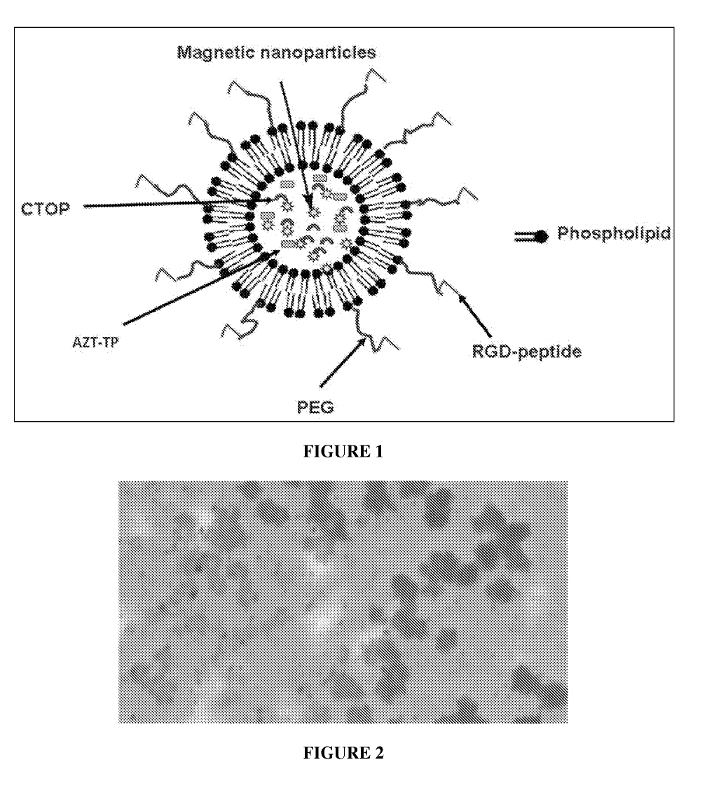

The magnetic nanoparticles bound to the therapeutic agents are encapsulated within the core of a liposome, which results in the formation of a drug-loaded magnetic liposome. The drug-loaded magnetic liposome can be further surface modified with specific affinity ligands (such as polyclonal/monoclonal antibodies, peptides, peptidomimetics, specific physiological ligands/analogues, for example) that target the magnetic liposome to the brain and also to circulating monocytes/macrophages. A specific example of a magnetic liposome as disclosed herein is shown in FIG. 1, where the antiviral agent AZT-TP and the specific μ-opioid receptor antagonist CTOP are bound to magnetic nanoparticles and encapsulated in a liposome having PEG and RGD-peptide on the outer surface of the liposome lipid bilayer.

Magnetic nanoparticles as used and disclosed herein include those that are approved by FDA as MRI contrast enhancement agents. Magnetic nanoparticles are magnetic materials that have no dimension greater than 5 μm, such as less than 4 μm, less than 3 μm, less than 2 μm, less than 1 μm, less than 500 nm, less than 100 nm, or are about 2 to about 15 nm. Magnetic materials include iron, cobalt, zinc, cadmium, nickel, gadolinium, chromium, copper, manganese, terbium, europium, gold, silver, platinum, oxides of any of the preceding, alloys of any of the preceding, or mixtures thereof. Specific examples of magnetic materials include, but are not limited to, iron oxide, superparamagnetic iron oxide, Fe₃O₄, Fe₂O₄, FeₓPt_{y}, CoₓPt_{y}, MnFeₓO_{y}, CoFeₓO_{y}, NiFeₓO_{y}, CuFeₓO_{y}, ZnFeₓO_{y}, and CdFeₓO_{y}, wherein x and y vary depending on the method of synthesis.

The magnetic nanoparticle optionally can have a coating over the magnetic material. Suitable coatings include dextran, chitosan, PLGA, dendrimers, amphiphilic polymers/bio-polymers (e.g. phospholipids and peptides), surfactants or chemical compounds with chelating properties for magnetic nanoparticles or high affinity adsorption (e.g. both chemisorption or physical adsorption) on the surface of magnetic nanoparticles, silicon oxide, silica, silica-PEG, mesoporous structures (silica or polymers or their combinations) for encapsulation of nanoparticles, or any other preferred combination of the above. In some cases, the coating is an amphiphilic polymer, for example, a phospholipid-PEG coating. Dextran-coated iron oxide nanoparticles have been approved for clinical human use by the FDA. Extensive studies on the biosafety, biodistribution, and metabolism of these superparamagnetic particles have shown that they are biocompatible and are degraded inside the body. The iron oxide is recycled and becomes raw materials for blood synthesis (hemoglobin).

The magnetic nanoparticles have a hydroxyl functional group on their surface, which allows for ionic binding of a therapeutic agent to the surface of the magnetic nanoparticle. This binding is reversible and allows for the bound drugs to be released, such as at a target site. The amount of therapeutic agent bound to the magnetic nanoparticle is controlled by the molar ratio of therapeutic agent to magnetic nanoparticle, incubation time for mixing of the two components, the pH of the incubation, the temperature of the incubation, and/or the buffers used during the incubation. For example, it is known that phosphates interact strongly with iron oxide particles, and therefore, the presence of phosphates during incubation will impact the amount of therapeutic agent bound to the magnetic nanoparticle surface. Binding of the therapeutic agent (or agents) to the magnetic nanoparticle provides a modified magnetic nanoparticle.

Contemplated therapeutic agents include, but are not limited to, natural enzymes, proteins derived from natural sources, recombinant proteins, natural peptides, synthetic peptides, cyclic peptides, antibodies, cytotoxic agents, immunoglobins, beta-adrenergic blocking agents, calcium channel blockers, coronary vasodilators, cardiac glycosides, antiarrhythmics, cardiac sympathomimetics, angiotensin converting enzyme (ACE) inhibitors, diuretics, inotropes, cholesterol and triglyceride reducers, bile acid sequestrants, fibrates, 3-hydroxy-3-methylgluteryl (HMG)-CoA reductase inhibitors, niacin derivatives, antiadrenergic agents, alpha-adrenergic blocking agents, centrally acting antiadrenergic agents, vasodilators, potassium-sparing agents, thiazides and related agents, angiotensin II receptor antagonists, peripheral vasodilators, antiandrogens, estrogens, antibiotics, retinoids, insulins and analogs, alpha-glucosidase inhibitors, biguanides, meglitinides, sulfonylureas, thiazolidinediones, androgens, progestogens, bone metabolism regulators, anterior pituitary hormones, hypothalamic hormones, posterior pituitary hormones, gonadotropins, gonadotropin-releasing hormone antagonists, ovulation stimulants, selective estrogen receptor modulators, antithyroid agents, thyroid hormones, bulk forming agents, laxatives, antiperistaltics, flora modifiers, intestinal adsorbents, intestinal anti-infectives, antianorexic, anticachexic, antibulimics, appetite suppressants, antiobesity agents, antacids, upper gastrointestinal tract agents, anticholinergic agents, aminosalicylic acid derivatives, biological response modifiers, corticosteroids, antispasmodics, 5-HT₄ partial agonists, antihistamines, cannabinoids, dopamine antagonists, serotonin antagonists, cytoprotectives, histamine H2-receptor antagonists, mucosal protective agent, proton pump inhibitors, H. pylori eradication therapy, erythropoieses stimulants, hematopoietic agents, anemia agents, heparins, antifibrinolytics, hemostatics, blood coagulation factors, adenosine diphosphate inhibitors, glycoprotein receptor inhibitors, fibrinogen-platelet binding inhibitors, thromboxane-A₂ inhibitors, plasminogen activators, antithrombotic agents, glucocorticoids, mineralcorticoids, corticosteroids, selective immunosuppressive agents, antifungals, drugs involved in prophylactic therapy, AIDS-associated infections, cytomegalovirus, non-nucleoside reverse transcriptase inhibitors, nucleoside analog reverse transcriptse inhibitors, protease inhibitors, anemia, Kaposi's sarcoma, aminoglycosides, carbapenems, cephalosporins, glycopeptides, lincosamides, macrolies, oxazolidinones, penicillins, streptogramins, sulfonamides, trimethoprim and derivatives, tetracyclines, anthelmintics, amebicides, biguanides, cinchona alkaloids, folic acid antagonists, quinoline derivatives, Pneumocystis carinii therapy, hydrazides, imidazoles, triazoles, nitroimidzaoles, cyclic amines, neuraminidase inhibitors, nucleosides, phosphate binders, cholinesterase inhibitors, adjunctive therapy, barbiturates and derivatives, benzodiazepines, gamma aminobutyric acid derivatives, hydantoin derivatives, iminostilbene derivatives, succinimide derivatives, anticonvulsants, ergot alkaloids, antimigrane preparations, biological response modifiers, carbamic acid eaters, tricyclic derivatives, depolarizing agents, nondepolarizing agents, neuromuscular paralytic agents, CNS stimulants, dopaminergic reagents, monoamine oxidase inhibitors, COMT inhibitors, alkyl sulphonates, ethylenimines, imidazotetrazines, nitrogen mustard analogs, nitrosoureas, platinum-containing compounds, antimetabolites, purine analogs, pyrimidine analogs, urea derivatives, anthracyclines, actinomycins, camptothecin derivatives, epipodophyllotoxins, taxanes, vinca alkaloids and analogs, antiandrogens, antiestrogens, nonsteroidal aromatase inhibitors, protein kinase inhibitor antineoplastics, azaspirodecanedione derivatives, anxiolytics, stimulants, monoamine reuptake inhibitors, selective serotonin reuptake inhibitors, antidepressants, benzisooxazole derivatives, butyrophenone derivatives, dibenzodiazepine derivatives, dibenzothiazepine derivatives, diphenylbutylpiperidine derivatives, phenothiazines, thienobenzodiazepine derivatives, thioxanthene derivatives, allergenic extracts, nonsteroidal agents, leukotriene receptor antagonists, xanthines, endothelin receptor antagonist, prostaglandins, lung surfactants, mucolytics, antimitotics, uricosurics, xanthine oxidase inhibitors, phosphodiesterase inhibitors, metheamine salts, nitrofuran derivatives, quinolones, smooth muscle relaxants, parasympathomimetic agents, halogenated hydrocarbons, esters of amino benzoic acid, amides (e.g. lidocaine, articaine hydrochloride, bupivacaine hydrochloride), antipyretics, hynotics and sedatives, cyclopyrrolones, pyrazolopyrimidines, nonsteroidal anti-inflammatory drugs, opioids, para-aminophenol derivatives, alcohol dehydrogenase inhibitor, heparin antagonists, adsorbents, emetics, opioid antagonists, cholinesterase reactivators, nicotine replacement therapy, vitamin A analogs and antagonists, vitamin B analogs and antagonists, vitamin C analogs and antagonists, vitamin D analogs and antagonists, vitamin E analogs and antagonists, vitamin K analogs and antagonists.

The therapeutic agents can be bound to the surface of the magnetic nanoparticles through a variety of means, including ionic interactions and covalent bonds. Ionic interactions between therapeutic agents and magnetic nanoparticles can occur between charged moieties or moieties capable of hydrogen bonding on the therapeutic agent and the magnetic nanoparticle. Covalent bonds between the therapeutic agent and the magnetic nanoparticle are preferably hydrolysable or releasable, such as ester bonds. For magnetic nanoparticles having hydroxyl groups on the surface, an ester bond can be formed between a therapeutic agent having a carboxylic acid moiety and the magnetic nanoparticle.

The modified magnetic nanoparticles can be incorporated into liposomes. The liposomes can provide biocompatibility for the modified magnetic nanoparticles, allowing the therapeutic agent to be delivered passed the BBB by application of an external magnetic field. The liposomes can be formed by known means, such as by mixture of phosphatidyl choline, phosphatidyl ethanolamine, and cholesterol. The liposomes optionally can further be formed with dihexadecyl phosphate (DHDP) and distearoyl phosphatidyl ethanolamine (DSPE). The liposomes further can include polyethylene glycol moieties. For example, the liposome can be prepared using a phosphatidyl moiety further having a PEG moiety. The PEG moiety, if present, extends from the surface of the liposome into the surrounding environment. The presence of a PEG moiety stabilizes the circulating half life of the liposome and further can provide a sustained release type formulation for the therapeutic agents bound to the magnetic nanoparticles in the liposome.

The disclosed liposomes can be formulated into pharmaceutical formulations suitable for parenteral administration. As used herein, “pharmaceutical formulation” is a composition of a pharmaceutically active drug, such as a biologically active protein, that is suitable for parenteral administration to a patient in need thereof and includes only pharmaceutically acceptable excipients, diluents, carriers and adjuvants that are safe for parenteral administration to humans at the concentrations used, under the same or similar standards as for excipients, diluents, carriers and adjuvants deemed safe by the Federal Drug Administration or other foreign national authorities. A pharmaceutical formulation may be in a ready-to-use solution form, concentrated form, or a lyophilized preparation that may be reconstituted with a directed amount of diluent suitable for parenteral injection such as water, salt solution, or buffer solution.

As used herein, “parenteral” administration of a medicament or formulation means administration to a subject by a route other than topical or oral (i.e., a non-topical and non-oral route). Examples of parenteral routes include subcutaneous, intramuscular, intravascular (including intraarterial or intravenous), intraperitoneal, intraorbital, retrobulbar, peribulbar, intranasal, intrapulmonary, intrathecal, intraventricular, intraspinal, intracisternal, intracapsular, intrasternal or intralesional administration. Parenteral administration may be, e.g., by bolus injection or continuous infusion, either constant or intermittent and/or pulsatile, and may be via a needle or via a catheter or other tubing.

FIG. 1 provides a schematic representation of the magnetic liposome, which would deliver the AZT-TP and CTOP across the BBB. The pegylation provides stability and increases the circulation half-life of the liposomes, by reducing its clearance by the reticuloendothelial system (RES). Additionally, an affinity ligand such as Arg-Gly-Asp; (RGD) peptide, antibodies, or the like, can be anchored to the PEG molecule on the surface of the magnetic liposomes, which can help in uptake of this liposome by phagocytic cells, especially monocytes and macrophages, through interaction with the receptors present on the surface of the monocytes. The uptake of the magnetic liposomes by monocytes, would make them magnetic cells thereby enhancing the drug loaded monocyte/macrophage transport across BBB under the influence of an external magnetic field.

The therapeutic agents that can be delivered using the disclosed magnetic liposomes include, but are not limited to, antiretrovirals, CNS cell based agonist and antagonist (such as dopamine, opioid, glutamate, GABA, and the like), neuropeptides, neuroprotective and neurotropic agents, and anticancer drugs.

The disclosed magnetic nanoparticles also can bind two or more therapeutic molecules belonging to different classes, such as an antiretroviral drug and agonist/antagonist of CNS cell surface receptors, and be incorporated into a liposome as disclosed herein. The resulting liposome can be targeted to a desired site magnetically by an external magnetic field. The magnetic nanoparticles of the present invention also have a property of acting as a contrast enhancing agent for magnetic resonance imaging (MRI). This provides a non-invasive method of monitoring nanoparticle accumulation, time course of drug delivery, and elimination of the magnetic nanoparticle from the brain.

The magnetic liposomes disclosed herein can be useful in the treatment of one or more CNS diseases (including, but not limited to, epilepsy, Parkinson's, Alzheimer's, and schizophrenia), neurobehavioral problems, NeuroAlDS, and drug addiction in affected individuals.

Not wishing to be bound by theory, the liposomes having modified magnetic nanoparticles can cross the BBB by two mechanisms:

(1) direct uptake under the influence of an externally applied magnetic field; and/or

(2) uptake of the liposome by circulating monocytes which then act as a carrier to transport the drug across the BBB.

Further description of the disclosed compositions and methods are provided below with respect to specific embodiments.

With the growing interest in nanotechnology, researchers have attempted nanoparticle based delivery platform for anti-retroviral drug. More recently, the use of magnetic nanoparticles has attracted significant importance in biomedical applications. Several in vitro and in vivo studies have been reported with magnetically guided drug targeting systems. However, no attempts have been reported using modified magnetic nanoparticles as a delivery system to treat simultaneously both drug addiction and neuro-AIDS. The liposomes having modified magnetic nanoparticles disclosed herein can provide a viable approach for simultaneously targeting of two or more drugs across the BBB.

A specific modified magnetic nanoparticle contemplated herein is a magnetic nanoparticle having two or more different therapeutic agents bound to the surface of the magnetic nanoparticle, for example, two different classes of therapeutics, such as active antiretroviral (5′-triphosphate of nucleoside reverse transcriptase inhibitor; NRTI) and a μ opioid receptor antagonist (such as CTOP). Encapsulation of 5′-triphosphates-NRTI, the biologically active form of antiviral NRTI, if able to cross the BBB, may be able to inhibit viral replication. CTOP, a μ opioid receptor antagonist, if able to cross the BBB, would be able to reduce opiate addiction and block the synergistic neurotoxicity caused by opiates and HIV proteins.

Neuropharmaceutics is the largest growth sector of the pharmaceutical industry, whose growth is hindered by the inability of most drugs to cross the BBB. Essentially, 100% of large molecule drugs (such as peptides, recombinant proteins, monoclonal antibodies, nucleic acid based drugs) and more than 98% of small drug molecules do not cross the BBB. In order to solve the BBB drug delivery problems, new innovative technologies are required in the area of pharmaceutics. The existing approaches to deliver drugs to brain includes: (1) trans-cranial drug delivery (2) trans-nasal drug delivery (3) transient disruption of BBB using hyperosomotic solutions and (4) lipidization of small molecules. Some of the technologies actively researched make use of endogenous BBB transporters to piggyback drugs across the BBB.

Drug Targeting Using Magnetic Nanoparticles and Liposomes

One of the types of nanoparticles that has gained increasing interest in recent years is the magnetic nanoparticle, which can comprise nano-sized iron oxide particles (such as Fe₃O₄ or γFe₂O₃) suspended in a carrier liquid. The use of magnetic nanoparticles contributes to a precise delivery of drugs to the exact site (e.g. inflammation, cancer and the like) by application of an external magnetic field. Magnetically guided drug targeting has been attempted in order to increase the efficacy and reduce the unpleasant side effects associated with cancer chemotherapy. This method of drug delivery involves immobilization of drug or radionuclide in biocompatible magnetic nano or microspheres. It aims to make chemotherapy more effective by increasing the drug concentration at the tumor site, while limiting the systemic drug concentration. The additional advantages come from such specific properties of magnetic nanoparticles as magnetic responsiveness and MRI visibility. Several investigators have previously shown that magnetic nanoparticles can be retained at tumor sites, after local administration combined with a locally applied external magnetic field, due to the “magnetic responsiveness” of the iron oxide core, thereby enabling magnetic targeting. However, liposomes are particularly attractive because they are biocompatibility and their vesicular structure can encapsulate a wide variety of hydrophilic and hydrophobic substances.

Liposomes of magnetic nanoparticles have been used for encapsulation of doxorubicin (DOX) for site specific chemotherapy in response to externally applied AC magnetic field. The result of this study showed that these liposomes can be specifically heated to 42° C. in a few minutes and during this, the encapsulated doxorubicin is massively released. Another in vivo studies for site specific targeting using liposomes incorporated with magnetic particles and adriamycin (ADR), showed that administration of magnetic ADR liposomes under an applied external magnetic force produced approximately 4-fold higher maximum ADR concentration in the tumor than did administration of ADR solution alone. These results suggested that systemic chemotherapy could effectively control the primary tumor without significant side effects, due to the targeting of magnetic ADR liposomes.

Magnetic nanoparticle based drug delivery and magnetic resonance imaging (MRI) systems also have been targeted to brain in animal models. However, no such studies have been attempted in human with respect to specific targeting of combination therapy of two or more therapeutic agents, such as ART plus opioid antagonist to brain. Thus, disclosed herein are magnetic nanocarriers for simultaneous delivery of two or more therapeutic agents across the BBB, such as ART and μ opioid receptor antagonist.

Administration of the antiviral NRTI (5′-Triphosphates-AZT) using the disclosed liposomes and magnetic nanoparticles may allow access across BBB of this antiviral to inhibit viral replication. Additionally, access of the CTOP μ opioid receptor blocker across the BBB (which does not cross BBB under normal condition) would reduce opiate addiction and block the synergistic neurotoxicity caused by opiates and HIV proteins. Additionally, administration of these liposomes can reduce the clinical toxicities induced by NRTIs. The modified magnetic nanoparticle delivery system can overcome many of the physiological barriers and deliver the drugs across the BBB, thereby reduce simultaneously NeuroAlDS and opiate addiction in HIV infected subjects who are opiate users.

Magnetic Liposomes as Drug Targeting Carriers

Characterization of the Magnetic Liposomes (ML): A typical transmission electron microscope (TEM) image of the prepared liposomes sample is shown in FIG. 2. The micrograph clearly shows the occurrence of relatively uniform, spherical shaped nanosized particles. The particles are well separated and their average size is of about 25 nm. TEM evidence of the occurrence of the phospholipid coating is obvious by transmission electron micrograph of samples stained with 0.5% uranyl acetate solution. The images show the occurrence of a thin layer surrounding each particle, which is attributed to the liposomes bilayer membrane.

The targeted approach of administering magnetic liposomes of therapeutic agents can provide better results than administration of normal liposomes for targeting of drugs to desired site. Accordingly, the potential of developing a similar approach to deliver multiple drugs to the target organ which could lead to the treatment of a variety of CNS disorders, including neuroAIDS in drug addicts exists.

The magnetite nanoparticles used herein are synthesized by chemical co-precipitation as described in Saiyed, et al., J Biotechnol. , 131:240-244 (2007), and specifically adsorb amphoteric hydroxyl (—OH) groups in aqueous media. These hydroxyl groups remain on the particles at a pH of 6 to 10. The presence of this functional group on the surface of magnetic particles allows binding of biomolecules/molecules based on ionic interactions. The binding of molecules to magnetic nanoparticles is reversible, which allow the bound drugs to be released at the target site. The drug bound particles are characterized by TEM for determination of size and shape. FTIR spectroscopy is performed on drug loaded nanoparticles to confirm the presence of the drug (in this instance, AZT-TP and CTOP). The amount of drug bound is determined by estimating the concentration of AZT-TP and CTOP in the unbound fraction using high performance liquid chromatography (HPLC). The binding procedure is standardized using different mole ratios of magnetic nanoparticles to drugs. Moreover, the mixture (MNP:drug) incubation time, temperature and pH of the buffer is optimized to give efficient binding of drugs to nanoparticles. It is well established that phosphate groups are known to interact strongly with Fe₃O₄ particles, it is anticipated that active form of NRTI (5′-triphosphate AZT) will bind strongly but reversibly to the magnetic nanoparticles of the present system, whereas CTOP being a peptide have charged groups (such as amino, carboxylate, guanidino, and the like) that can bind to magnetic nanoparticles by ionic interactions or can be coupled covalently via an amide or ester bond.

Magnetic liposomes (MLs) as disclosed herein are prepared by reverse phase evaporation method using various molar ratios of phosphatidyl choline (PC), phosphatidyl ethanolamine (PE), and cholesterol, as described in Szoka, et al., Proc. Natl. Acad. Sci. USA, 75:4194-4198 (1978). The MLs are pegylated by incorporation of PEG-distearoylphosphatidylethanolamine (DSPE) during the preparation of the lipid bilayer. The lipid bilayer is rehydrated in the presence of magnetic nanoparticles bound to 5′-triphosphate AZT and CTOP resulting in the encapsulation of the drugs within the core of the liposomes and thus forming a ML. RGD peptide is anchored to the surface of the MLs by covalent coupling to the PEG moiety. Anchoring of affinity ligands, such as RGD peptide, is described in Jansons, et al., Anal. Biochem., 111:54-59 (1980).

In Vitro Assessment of Transport of Magnetic Nanocarrier Across the Blood Brain Barrier

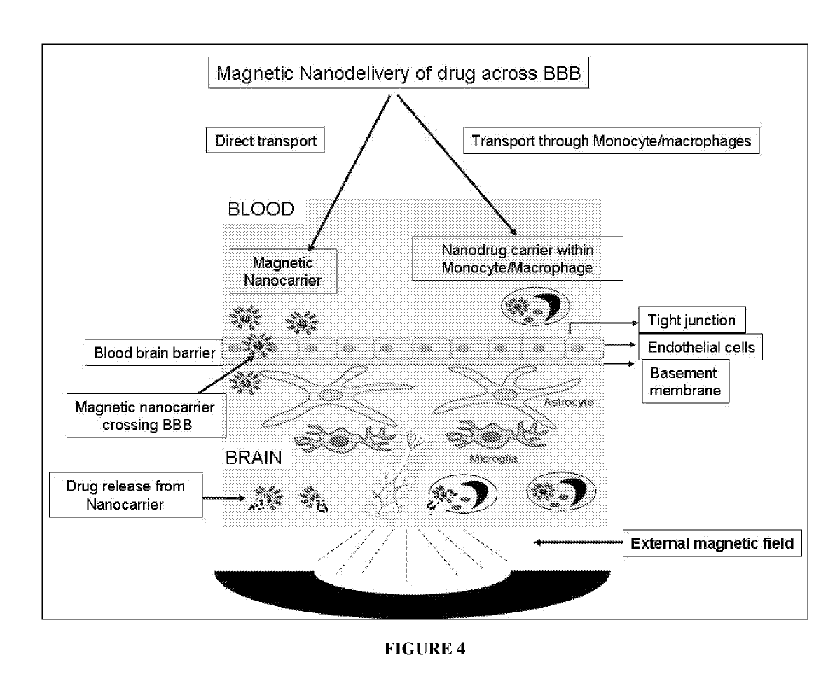

There are two mechanism hypothesized for the transport of magnetic liposomes across the BBB as depicted in FIG. 4: 1) Direct uptake and delivery of the magnetic liposome from the circulation into the CNS under the influence of an external magnetic field of appropriate strength, 2) Uptake of the magnetic liposome by circulating monocytes/macrophages (due to the presence of affinity ligand, RGD peptide that interacts with the integrin receptors present on the surface of monocytes/macrophages) followed by the entry of magnetized monocyte/macrophages into the brain. Monocytes having magnetic liposomes as described herein can be prepared as described in Gandhi, et al., AIDS Res. Human Retrovir., 25(7):691-699 (2009). After the delivery of the liposome into the brain, the drugs are released over a period of time providing a sustained release effect, which may help to reduce the toxicity and increase efficacy of the ART and CTOP.

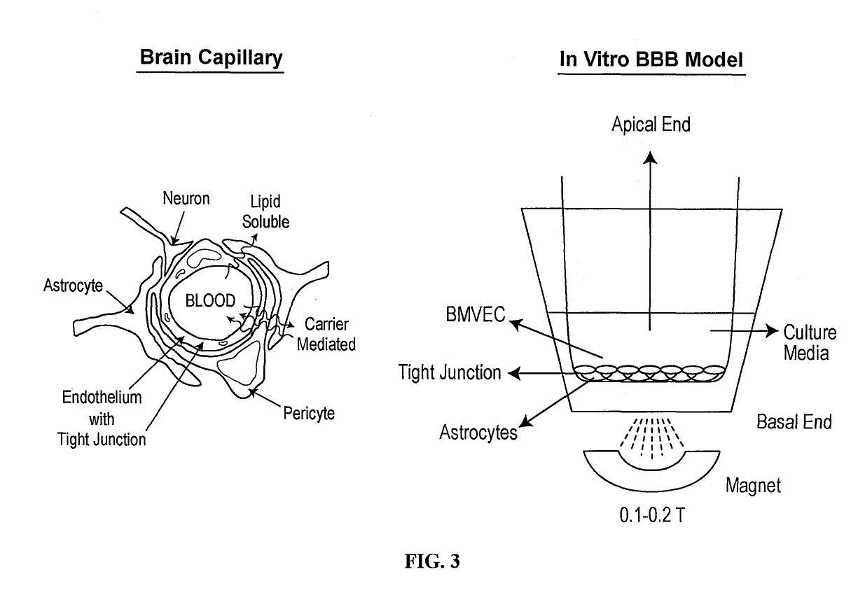

The BBB model (schematically shown in FIG. 3) comprises two-compartment wells in a culture plate with the upper compartment separated from the lower by a Cyclopore polyethylene terephthalate membrane (Collaborative Biochemical Products, Becton Dickinson, San Jose, Calif.) containing 2×10⁶ pores/cm² (pore diameter 3 μm). The brain microvascular endothelial cells (BMVEC) are grown to confluency on the upper side while a confluent layer of human astrocytes is grown on the underside. The BBB model is used for experiments at least ≧5 days after cell seeding. The intactness of BBB is determined by measuring the transendothelial electrical resistance (TEER) using Millicell ERS microelectrodes (Millipore, Bedford, Mass.) and inulin permeability.

### EXAMPLES

Magnetic Nanoparticle-AZTTPs

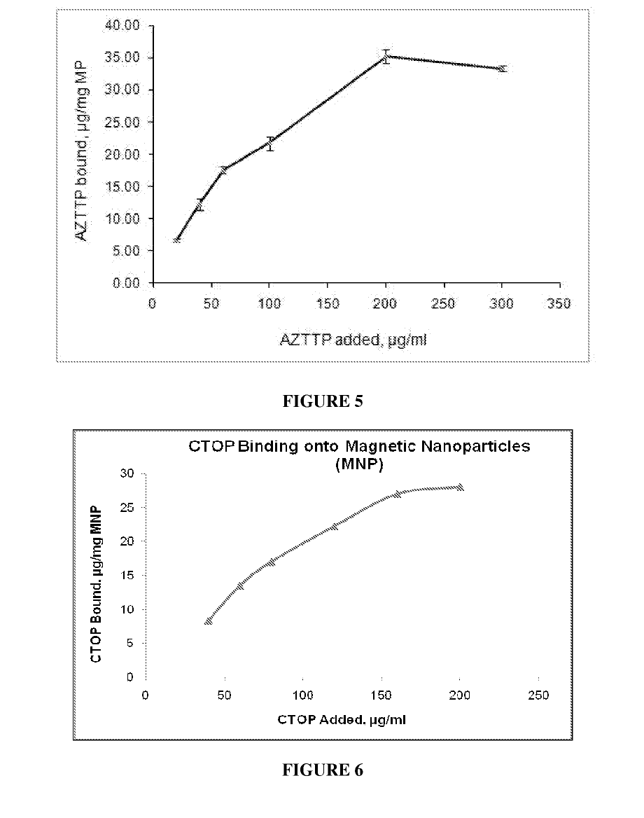

NRTIs remain as an important component of the combination antiretroviral therapy (ART). However, the low conversion of NRTIs into their active nucleoside 5′-triphosphate (NTP) form results in the accumulation of NRTIs that leads to the development of drug resistance, toxicity, and ultimately compromising the effectiveness of this therapy. Therefore, target specific delivery of NRTI in this active triphosphate form through an effective carrier provides significant therapeutic benefit and also reduces clinical toxicities associated with high doses of NRTIs. 3′-Azido-3′-Deoxythymidine-5′-triphosphate (AZT-TP) an active form of AZT was directly bound to magnetic nanoparticles (FIG. 5). The mechanism of direct binding is due to the strong interaction of triphosphate groups of AZTTP with the —OH group of magnetic nanoparticles. This ionic interaction also helps to neutralize the electronegative charge of AZTTP and make their mass transport easier across the cellular barriers.

CTOP peptide was bound to magnetic nanoparticles in the presence of carbodiimide as a coupling agent (see FIG. 6). CTOP can be used for treatment of opiate addiction and to inhibit the synergistic neurotoxicity caused by opioid and HIV-1.

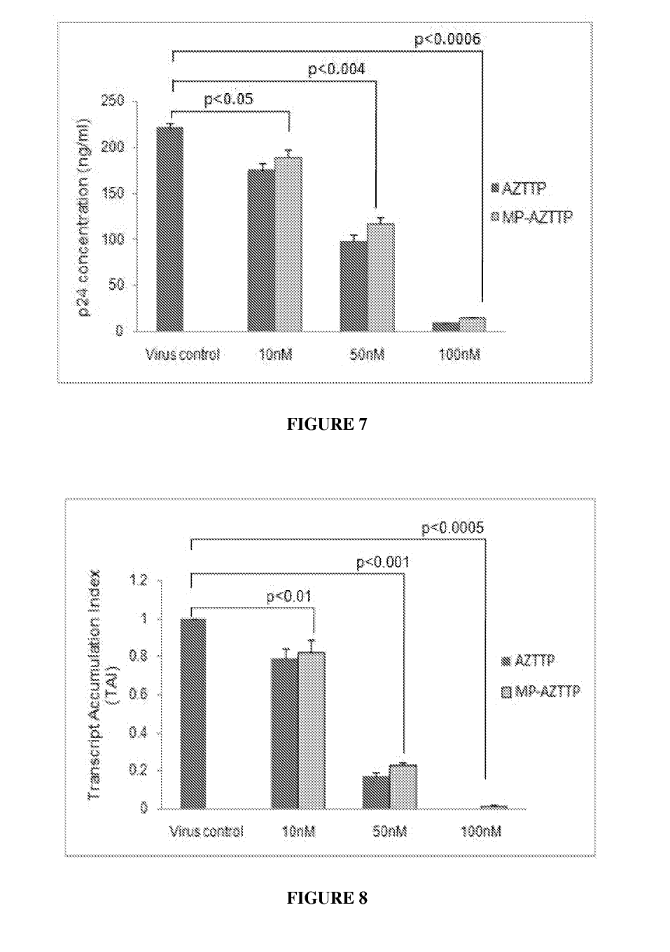

The efficacy of magnetic nanoparticle-AZTTP was evaluated in an in vitro model system using HIV infected peripheral blood mononuclear cells (PBMCs). A dose dependent decrease in p24 antigen production was evident at lOnM (183±8.8 ng/ml, 18% decrease, p<0.05), 50 nM (107±7.04 ng/ml, 52% decrease, p<0.004) and 10nM (15+0.9 ng/ml, 93% decrease, p<0.0006) of MP-AZTTP treatment by HIV-1 infected PBMCs compared to the untreated control cultures (221.4±4.5) (FIG. 8). The anti-HIV activity of free AZTTP (positive control) and MP-AZTTP was found to be similar under identical culture conditions at various doses. In addition to p24 antigen decrease, MP-AZTTP treatment also suppressed LTR-R/U5 gene expression in a dose dependent manner (FIG. 7). These results show that AZTTP bound to magnetic nanoparticles (MP-AZTTP) is able to exert significant anti-HIV effects comparable to free AZTTP. Therefore, binding of AZTTP to magnetic nanoparticles have no effect on its biological activity.

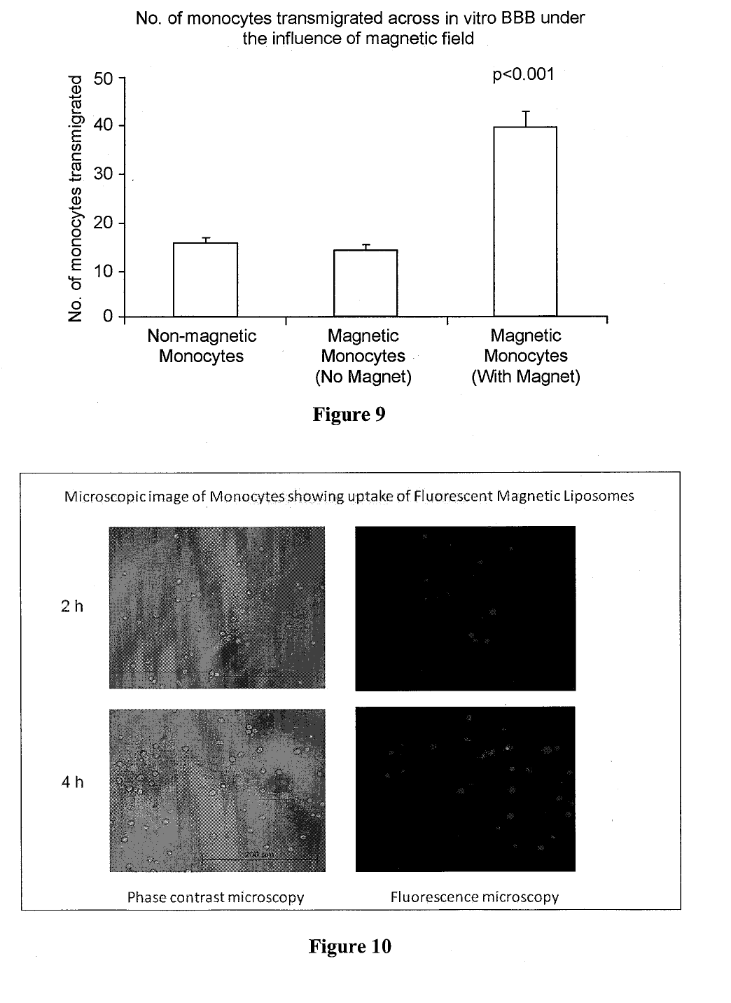

Transport of magnetic monocytes across the in vitro BBB: AZTTP loaded fluorescent magnetic liposomes were prepared and incubated with monocytes so as to allow their uptake by monocytes. FIG. 10 shows significant uptake of the magnetic liposomes by the monocytes after 2 and 4 hours of incubation. After incubation, monocytes were washed thoroughly to remove unencapsulated magnetic nanocarrier. The magnetic monocytes were then loaded in upper chamber of an in vitro BBB with a magnet placed underneath. The transport of the monocytes across the BBB was monitored by measuring the number of transmigrated monocytes in the lower chamber after 4 h. FIG. 9 shows that magnetic monocytes were able to transmigrate across the BBB with 2-3 fold higher rates than non-magnetic monocytes.

The foregoing describes and exemplifies the invention but is not intended to limit the invention defined by the claims which follow. All of the methods disclosed and claimed herein can be made and executed without undue experimentation in light of the present disclosure. While the materials and methods of this invention have been described in terms of specific embodiments, it will be apparent to those of skill in the art that variations may be applied to the materials and/or methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved.

1. A method of delivering at least one therapeutic agent to a patient across a blood brain barrier comprising
administering to the patient a liposome comprising magnetic nanoparticles and at least one therapeutic agent bound to a surface of the magnetic nanop article.

2. The method of claim 1, wherein the therapeutic agent is reversibly bound to the surface of the magnetic nanoparticle.

3. The method of claim 2, wherein the therapeutic agent is reversibly bound to the surface of the magnetic nanoparticle by an ionic interaction between the therapeutic agent and the magnetic nanoparticle.

4. The method of claim 2, wherein the therapeutic agent is reversibly bound to the portion of the surface of the magnetic nanoparticle by a hydrolysable covalent bond between the therapeutic agent and the magnetic nanoparticle.

5. The method of claim 4, wherein the hydrolysable covalent bond comprises an ester bond.

6. The method of claim 1, wherein the magnetic nanoparticle comprises iron oxide.

7. The method of claim 1, wherein two or more therapeutic agents are bound to the surface of the magnetic nanoparticle.

8. The method of claim 1, wherein the liposome comprises a first magnetic nanoparticle having a first therapeutic agent bound to the first magnetic nanoparticle surface and a second magnetic nanoparticle having a second therapeutic agent bound to the second magnetic nanoparticle surface.

9. The method of claim 1, wherein the liposome comprises a first magnetic nanoparticle having a first therapeutic agent bound to the first magnetic nanoparticle surface and a second therapeutic agent.

10. The method of claim 7, wherein a first therapeutic agent comprises an antiretroviral and a second therapeutic agent comprises a t-opioid receptor antagonist.

11. The method of claim 10, wherein the antiretroviral comprises 5′-triphosphate- azidothymidine (AZT-TP) and the μ-opioid receptor antagonist comprises CTOP.

12. The method of claim 1, wherein the patient has Neuro-AIDS, an opiate addiction, or both.

13. The method of claim 12, wherein the patient suffers from both Neuro-AIDS and an opiate addiction.

14. The method of claim 1, wherein the patient suffers from a central nervous system disorder.

15. The method of claim 14, wherein the central nervous system disorder is selected from the group consisting of a brain carcinoma, epilepsy, Parkinson's disease, Alzheimer's disease, schizophrenia, alcohol addiction, opioid addiction, cocaine addiction, and methamphetamine addiction.

16. The method of claim 1, further comprising exposing the patient to a magnetic field to deliver the therapeutic agent across the BBB.

17. The method of claim 1, further comprising detecting the magnetic nanoparticles by magnetic resonance imaging (MRI).

18. The method of claim 1, wherein the liposome is formulated into a formulation suitable for parenteral administration.

19. The method of claim 18, wherein the parenteral administration is intravenous administration.

20. The method of claim 18, wherein the formulation provides a sustained release of the therapeutic agent.
